SEQUENCE LISTING (1 sequence)

SEQ ID NO: 1 - DNA, 20 bp, Artificial Sequence
  Synthetic
tccatgacgttcctgacgtt

(19) United States
(12) Patent Application Publication
(10) Pub. No.: US 2008/0008652 A1
(43) Pub. Date: Jan. 10, 2008

(54) Methods and compositions for mediation of immune responses and adjuvant activity

(75) Inventors: Gabriel Nunez, Ann Arbor, MI (US); Thirumala-Devi Kanneganti, Ann Arbor, MI (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 11/651,808

(22) Filed: Jan. 10, 2007

Related U.S. Application Data
(60) Provisional application No. 60/757,649, filed on Jan. 10, 2006.

(51) Int. Cl. A61K 49/00; C12Q 1/02; C12Q 1/34; C12Q 1/68

(52) U.S. Cl. 424/9.2; 435/18; 435/29; 435/6

(57) ABSTRACT

The present invention relates to methods and compositions for modulating immune responses and adjuvant activity. In particular, the present invention provides methods and compositions for screening for compounds that modulate cryopyrin signaling.

This application claims priority to provisional patent application 60/757,649, filed Jan. 10, 2006, which is herein incorporated by reference in its entirety.

This invention was made with government support under grant No. RO1 AI063331 from the National Institutes of Health. The government has certain rights in the invention.

### FIELD OF THE INVENTION

The present invention relates to methods and compositions for modulating immune responses and adjuvant activity. In particular, the present invention provides methods and compositions for screening for compounds that modulate cryopyrin signaling.

### BACKGROUND OF THE INVENTION

There are more than 80 different known autoimmune and inflammatory diseases that are characterized by abnormal triggering of an inflammatory response that attacks the host's own organs or tissues.

Examples of autoimmune and inflammatory diseases include rheumatoid arthritis, inflammation of the heart (myocarditis), asthma, inflammatory bowel disease such as Crohn's disease and ulcerative colitis, systemic lupus erythematosus, rheumatic fever, autoimmune hemolytic anemia, idiopathic thrombocytopenic purpura, and postviral encephalomyelitis.

There are a number of treatment options for inflammatory diseases including medications, rest and exercise, and surgery. The type of treatment depends on several factors, including the type of disease, the person's age, type of medications he or she is taking, overall health, medical history and severity of symptoms. Common medications include nonsteroidal anti-inflammatory drugs (NSAIDs such as aspirin, ibuprofen or naproxen), corticosteroids (such as prednisone), salicylates, antimalarial medications (such as hydroxychloroquine), and other medications including gold, methotrexate, sulfasalazine, penicillamine, cyclophosphamide, infliximab, etanercept and cyclosporine. However, many of the existing treatments have unpleasant side effects and are not effective.

Clearly there is a great need for identification of the molecular basis of inflammatory disease. There is also a need for new, more effective treatments with fewer side effects than the existing treatments.

### SUMMARY OF THE INVENTION

The present invention relates to methods and compositions for modulating immune responses and adjuvant activity. In particular, the present invention provides methods and compositions for screening for compounds that modulate cryopyrin signaling.

Accordingly, in some embodiments, the present invention provides methods for screening for compounds that modulate immune responses in a cell, tissue, or organism (e.g., by modulating cryopyrin signaling (e.g., via activation of caspase-1)). The present invention further provides methods of modulating immune responses and adjuvant activity (e.g., using the compounds identified in drug screening assays).

For example, in some embodiments, the present invention provides a method of screening compounds, comprising contacting a cell expressing cryopyrin with a test compound; and comparing the level of caspase-1 activation in the presence of the test compound to the level in the absence of the test compound. In some embodiments, the level of caspase-1 activation is higher in the presence of the test compound relative to the level in the absence of the test compound. In some embodiments, the test compound is an adjuvant. In other embodiments, the test compound is a drug. In further embodiments, the test compound is a nucleic acid, a nucleotide, a nucleoside, a purine or a modified form or mimetic thereof. In some embodiments, measuring the level of caspase-1 activation comprising measuring the level of a cytokine (e.g., IL-1β or IL-18). In some embodiments, the cell is in a non-human mammal.

In other embodiments, the present invention provides a method of modulating an immune response, comprising contacting a cell with an immune modulator and a cryopyrin modulator. In some embodiments, the cryopyrin modulator stimulates cryopyrin expression or activity. In some embodiments, the cell is cryopyrin deficient and the cryopyrin modulator is a cryopyrin nucleic acid or a cryopyrin polypeptide.

In other embodiments, the immune modulator is an adjuvant (e.g., Imiquimod or Resquimod). In some embodiments, the method further comprises administering a vaccine to the cell. In preferred embodiments, following administration of the vaccine to the cell, production of vaccine specific IgG is stimulated in the cell. In still other embodiments, the immune modulator is a pathogen (e.g., a bacteria or bacterial RNA). In some embodiments, the immune modulator activates a pro-inflammatory response. In some embodiments, secretion of IL-1β and IL-18 is stimulated in the cell. In some embodiments, caspase-1 is activated in the cell.

In some embodiments, the cell is in an organism. In some embodiments, the organism is a non-human mammal (e.g., a non-human mammal that lacks a functional cyropyrin gene). In other embodiments, the organism is a human.

Other embodiments of the invention are described in the description and examples below.

### DESCRIPTION OF THE FIGURES

FIG. 1 shows that cryopyrin is required for IL-1β and IL-18 secretion in response to imidazoquinoline compounds R837 and R848. a-b, Peritoneal macrophages (a) and BMDM (b) from WT (black bars) or cryopyrin−/− mice (white bars) were stimulated with the indicated stimuli for 24 h. c, Peritoneal macrophages (left panels) and BMDM (right panels) were stimulated with the indicated concentrations of R837 for 24 h.

FIG. 2 shows caspase-1 processing and NF-κB activation in mutant macrophages stimulated with R837 or R848. a-b, BMDM from WT and cryopyrin−/− mice (a) or WT and TLR7−/− mice (b) were stimulated with R837 (5 μg ml-1) for the indicated times. c-g, BMDM from WT and the indicated mutant mice were stimulated with R837 (5 μg ml-1) or R848 (5 μg ml-1) (c, e, f, g), LPS, lipid A (LA), or MDP (10 μg/ml-1) (d).

FIG. 3 shows that Cryopyrin is essential for activation of caspase-1 in response to bacterial RNA. a, BMDM from WT (black bars) or cryopyrin−/− mice (white bars) were stimulated with the indicated stimuli for 24 h. b, BMDM from WT and cryopyrin−/− mice were stimulated with purified RNA from E. coli for the indicated times and then pulsed transiently with ATP for 30 min. c, BMDM from WT and cryopyrin−/− mice were stimulated with purified RNA from various bacteria for 3 h in the absence and then pulsed transiently with ATP for 30 min. d, BMDM from WT and cryopyrin−/− mice were stimulated with purified total RNA with (+) and without (−) RNAse digestion from various bacteria and mouse liver for 3 h. e, BMDM from WT and various mutant mice were stimulated with purified RNA from E. coli or mouse liver for 3 h and then pulsed transiently with ATP for 30 min.

FIG. 4 shows that R837 and LPS cooperate for the production of pro-inflammatory cytokines in a cryopyrin-dependent manner. a, BMDM from WT or cryopyrin−/− mice (KO) were co-stimulated with LPS and the indicated stimuli for 24 hrs, or left unstimulated (Unstim). b-d, Groups of WT mice and cryopyrin−/− mice (KO) (n=7) were co-injected with LPS (200 μg), R837 (200 μg) or LPS plus R837 (200 μg each) and levels of IL-1β (a), IL-6 (b) or TNFα (c) in serum were determined by ELISA at the indicated times.

FIG. 5 shows impaired antigen-specific immunoglobulin production in cryopyrin−/− mice immunized with R837. a, WT (n=7) and cryopyrin−/− (n=7) mice (8-12 weeks old) were immunized with R837 (100 μg) and HSA (50 μg) or HSA alone and bled 2 weeks after immunization. b, The immunized mice were boosted 3 weeks later with HSA (20 μg) and R837 (10 μg) and bled one week later.

FIG. 6 shows the generation of cryopyrin deficient mice. a, Schematic representation of the genomic cryopyrin locus, gene-targeting construct, and the targeted cryopyrin allele. b, The location of the primers used for PCR is indicated by arrows in a. c, Expression of Cryopyrin by RT-PCR analysis of spleen tissue from WT (cryo+/+) and cryopyrin−/− mice. d, Western blot analysis of Cryopyrin expression in LPS-stimulated Cryo+/+ and Cryo−/− macrophages (upper panel). The same blot was reprobed for β-actin antibody to control for protein loading (lower panel).

FIG. 7 shows that cryopyrin is dispensable for IL-1β secretion in response to TLR2 and TLR4 agonists.

FIG. 8 shows that Cryopyrin is required for IL-1β secretion in response to imidazoquinoline compounds R837 and R848 plus ATP.

FIG. 9 shows that cryopyrin is dispensable for IFN-α secretion in response to viruses: influenza-WSN, vesicular stomatitis virus (VSV) and herpes simplex virus-1 (HSV-1). a-b, Bone-marrow-derived dendritic cells (a) and macrophages (b) from WT (black bars) or cryopyrin−/− mice (white bars) were stimulated with indicated viruses for 24 h and cell-free supernatants were analyzed by ELISA for production of IFN-α.

FIG. 10 shows NF-κB activation in MyD88-deficient macrophages stimulated with R-837.

FIG. 11 shows IL-1β secretion and caspase-1 activation processing in cryopyrin+/+, cryopyrin+/− and cryopyrin−/− mice. a, BMDM from WT (black bars), cryopyrin+/− mice (gray bars) or Cryopyrin−/− mice (white bars) were stimulated with the indicated stimuli for 24 h and cell-free supernatants were analyzed by ELISA for production of IL-1β. b, BMDM were stimulated with indicated stimuli for 3 h and cells were pulsed transiently with ATP for 30 min.

FIG. 12 shows that endosomal acidification is not required for IL-1β secretion in response to LPS, R837 and E. coli RNA.

FIG. 13 shows IL-1β secretion in ASC, MyD88 and TLR7 deficient macrophages. a-c, BMDM from WT and ASC−/− (a), MyD88−/− (b) and TLR7−/− (c) mice were treated with indicated stimuli for 24 h and cell-free supernatants were analyzed by ELISA for production of IL-1β.

FIG. 14 shows that wild-type and cryopyrin−/− mice show comparable susceptibility to LPS-induced endotoxic shock. Indicated numbers of wild-type and cryopyrin−/− mice (8 to 12 weeks old) were challenged with 200 μg (a) or 400 μg (b) of LPS and the survival over time of each mouse genotype was plotted.

FIG. 15 shows CpG adjuvant activity in WT and cryopyrin−/− mice. a, WT (n=5) and cryopyrin−/− (n=5) mice (8-12 weeks old) were immunized with CpG-ODN (100 μg) and HSA (50 μg), boosted 3 weeks later with HSA (20 μg) and CpG-ODN (20 μg) and bled one week later.

### DEFINITIONS

To facilitate understanding of the invention, a number of terms are defined below.

As used herein, the term “immune modulator” refers to any compound, molecule or other substance that alters (e.g., inhibits or stimulates) a cell or organism's immune system. In some embodiments, immune modulators include, but are not limited to, adjuvants (e.g., Imiquimod and Resiquimod) and pathogens (e.g., pathogen RNA). Immune response may be measured using any suitable method, include but not limited to, those disclosed herein. For example, in some embodiments, the activity of immune modulators of the present invention is measured by assaying production of interleukins (e.g., IL-1β or IL-18) or immunoglobulins (e.g., IgGs specific for a given antigen).

And used herein, the term “cryopyrin” is used interchangeably with the terms “Nalp3,” “Pypaf1,” and “Caterpiller 1.1” to refer to the product of the CIAS1 gene.

As used herein, the term “cryopyrin modulator” refers to any compound, molecule or other substance that alters the activity of cyropyrin. In some embodiments, cyropyrin modulators stimulate or inhibit expression of cyropyrin nucleic acids or polypeptides. In other embodiments, cyropyrin modulators stimulate or inhibit cryopyrin activity (e.g., by altering cryopyrin signaling activity).

As used herein, the term “activates caspase-1,” when used in reference to any molecule that activates caspase-1, refers to a molecule (e.g., a protein) that induces the activity of caspase-1 through a cell signaling pathway. Assays for determining if a molecule activates caspase-1 utilize, for example, secretion of cytokines (e.g., IL-1β or IL-18). Suitable assays include, but are not limited to, those described in the Experimental section below.

The term “apoptosis” means non-necrotic cell death that takes place in metazoan animal cells following activation of an intrinsic cell suicide program. Apoptosis is a normal process in the development and homeostasis of metazoan animals. Apoptosis involves characteristic morphological and biochemical changes, including cell shrinkage, zeiosis, or blebbing, of the plasma membrane, and nuclear collapse and fragmentation of the nuclear chromatin, at intranucleosomal sites, due to activation of an endogenous nuclease.

As used herein, the term “mimetic” refers to a small molecule compound that mimics the binding or interaction of a ligand with its target. For example, some cryopyrin ligands are mimetics of pathogen RNA (e.g., nucleotides, nucleosides, or purines).

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “peptide” refers to a polymer of two or more amino acids joined via peptide bonds or modified peptide bonds. As used herein, the term “dipeptides” refers to a polymer of two amino acids joined via a peptide or modified peptide bond.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions with its various ligands and/or substrates.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a polypeptide includes, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets that specify stop codons (i.e., TAA, TAG, TGA).

As used herein, the term “purified” or “to purify” refers to the removal of contaminants from a sample. For example, cryopyrin antibodies are purified by removal of contaminating non-immunoglobulin proteins; they are also purified by the removal of immunoglobulin that does not bind cryopyrin. The removal of non-immunoglobulin proteins and/or the removal of immunoglobulins that do not bind cryopyrin results in an increase in the percent of cryopyrin-reactive immunoglobulins in the sample. In another example, recombinant cryopyrin polypeptides are expressed in bacterial host cells and the polypeptides are purified by the removal of host cell proteins; the percent of recombinant cryopyrin polypeptides is thereby increased in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “transgene” as used herein refers to a foreign gene that is placed into an organism by introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene. The term “autologous gene” is intended to encompass variants (e.g., polymorphisms or mutants) of the naturally occurring gene. The term transgene thus encompasses the replacement of the naturally occurring gene with a variant form of the gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. As used herein, the term “sample” is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present invention. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the term “response,” when used in reference to an assay, refers to the generation of a detectable signal (e.g., accumulation of reporter protein, increase in ion concentration, accumulation of a detectable chemical product).

As used herein, the term “nucleic acid binding protein” refers to proteins that bind to nucleic acid, and in particular to proteins that cause increased (i.e., activators or transcription factors) or decreased (i.e., inhibitors) transcription from a gene.

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

### DETAILED DESCRIPTION OF THE INVENTION

The present invention relates to methods and compositions for modulating immune responses and adjuvant activity. In particular, the present invention provides methods and compositions for modulating cryopyrin activity.

Missense mutations in the CIAS1 gene cause three autoinflammatory disorders: familial cold autoinflammatory syndrome, Muckle-Wells syndrome, and neonatalonset multiple-system inflammatory disease (Stojanov et al., Curr Opin Rheumatol 17, 586-99 (2005)). Cryopyrin (also called Nalp3, Pypaf1 and Caterpiller 1.1), the product of CIAS1, is a member of the NOD-LRR protein family that has been implicated in cytosolic recognition of microbial ligands and activation of host defense signalling pathways (Inohara et al., Annu Rev Biochem 74, 355-83 (2005); Athman et al., Curr Opin Microbiol 7, 25-32 (2004)). Cryopyrin forms a multi-protein complex containing the adaptor ASC and caspase-1 termed “the inflammasome” which promotes caspase-1 activation and processing of prointerleukin (IL)-1β4. Experiments conducted during the course of development of the present invention demonstrated that signaling through cryopyrin (e.g., activation of caspase-1) was necessary for modulating certain immune responses and inflammosome responses.

Accordingly, in some embodiments, the present invention provides methods of identifying modulators of cryopyrin signaling (e.g., compounds that activate caspase-1). Such compounds find use in the modulation of immune responses. The present invention further provides methods of altering cryopyrin signaling. The below description provides non-limiting examples of drug screening and therapeutic applications of altering cryopyrin signaling. One skilled in the relevant art recognizes that other applications are within the scope of the present invention.

I. Drug Screening Using Cryopyrin

The present invention provides methods and compositions for using cryopyrin as a target for screening drugs that can alter, for example, inflammatory response, anti-viral response, anti-tumor response or adjuvant activity to enhance antigen specific antibody production. For example, drugs that induce or inhibit cryopyrin induced activation of caspase-1 can be identified by screening for compounds that interact with cryopyrin or cryopyrin modulating proteins, regulate cryopyrin gene expression or activate cryopyrin signaling via caspase-1 activation. In other embodiments, drug screening assays identify ligands or modulators of cryopyrin signaling via caspase-1 for use in the treatment of inflammatory disease or in adjuvant formulations.

In some embodiments, test compounds mimic natural ligands of cryopyrin (e.g., RNA). For example, in some embodiments, test compounds are nucleotides, nucleosides, purines, or other RNA mimetics. In other embodiments, test compounds are based on the structure of R837 or R848, which have been shown to activate caspase-1 via cryopyrin (See experimental section below).

The present invention is not limited to the test compounds described above. Test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In some embodiments, drug screens utilize cells (e.g., in vitro, ex vivo, or in vivo) that express cryopyrin. The cells are contacted with test compounds and the ability of the test compounds to activate caspase-1 is measured. Caspase-1 activation may be measuring using any suitable direct or indirect method. In some embodiments, caspase-1 activation is assayed my measuring the level of IL-1β or IL-18. An increase in levels of IL-1β or IL-18 is indicative of activation of caspase-1. In some embodiments, candidate compounds are those that increase the levels of IL-1β or IL-18 relative to the levels in the absence of the test compound.

In other embodiments, macrophages are isolated from wild-type and cryopyrin deficient mice. The macrophages are contacted with test compounds and the level of caspase-1 activation is measured (e.g., by immunoblotting, reporter substrates or levels of IL-1β or IL-18). Preferred test compounds are those that activate caspase-1 in wild-type, but not cryopyrin deficient macrophages.

In other embodiments, drug screens are used to identify compounds that alter the ability of cryopyrin to interact with binding partners. It is contemplated that binding assays are useful for screening for compounds that block or enhance cryopyrin binding to cryopyrin binding partners. The binding need not employ full-length cryopyrin binding partner and cryopyrin. Indeed, portions of cryopyrin binding partner and cryopyrin may be utilized in the binding assays.

In other embodiments, the present invention provides methods of screening for compounds that interact with cryopyrin or cryopyrin signaling partners and thus alter caspase-1 activation. In some embodiments, wild-type cryopyrin or a fragment thereof is utilized. In other embodiments, cryopyrin containing one or more variations (e.g., mutations or polymorphisms) is utilized.

In one screening method, the two-hybrid system is used to screen for compounds capable of altering (e.g., enhancing or inhibiting) cryopyrin function(s) (e.g., activation of caspase-1 or secretion of IL-1β or IL-18 or antigen specific IgG) in vitro or in vivo. In one embodiment, a GAL4 binding site, linked to a reporter-gene such as lacZ, is contacted in the presence and absence of a candidate compound with a GAL4 binding domain linked to a cryopyrin fragment and a GAL4 transactivation domain II linked to an cryopyrin ligand or fragment thereof. Expression of the reporter gene is monitored and a decrease in the expression is an indication that the candidate compound inhibits the interaction of cryopyrin with its ligand.

In another screening method, candidate compounds are evaluated for their ability to alter cryopyrin signaling by contacting cryopyrin or other proteins in cryopyrin signaling pathways, or fragments thereof, with the candidate compound and determining binding of the candidate compound to the peptide. The protein or protein fragments is/are immobilized using methods known in the art such as binding a GST-cryopyrin fusion protein to a polymeric bead containing glutathione. A chimeric gene encoding a GST fusion protein is constructed by fusing DNA encoding the polypeptide or polypeptide fragment of interest to the DNA encoding the carboxyl terminus of GST (See e.g., Smith et al., Gene 67:31 [1988]). The fusion construct is then transformed into a suitable expression system (e.g., E. coli XA90) in which the expression of the GST fusion protein can be induced with isopropyl-β-D-thiogalactopyranoside (IPTG). Induction with IPTG should yield the fusion protein as a major constituent of soluble, cellular proteins. The fusion proteins can be purified by methods known to those skilled in the art, including purification by glutathione affinity chromatography. Binding of the candidate compound to the proteins or protein fragments is correlated with the ability of the compound to disrupt the signal transduction pathway and thus regulate cryopyrin physiological effects (e.g., inflammatory disease).

In another screening method, one of the components of the cryopyrin signaling system, such as cryopyrin or a fragment of cryopyrin, is immobilized. Polypeptides can be immobilized using methods known in the art, such as adsorption onto a plastic microtiter plate or specific binding of a GST-fusion protein to a polymeric bead containing glutathione. For example, GST-cryopyrin is bound to glutathione-Sepharose beads. The immobilized peptide is then contacted with another peptide with which it is capable of binding in the presence and absence of a candidate compound. Unbound peptide is then removed and the complex solubilized and analyzed to determine the amount of bound labeled peptide. A decrease in binding is an indication that the candidate compound inhibits the interaction of cryopyrin with the other peptide. A variation of this method allows for the screening of compounds that are capable of disrupting a previously-formed protein/protein complex. For example, in some embodiments a complex comprising cryopyrin or a cryopyrin fragment bound to another peptide is immobilized as described above and contacted with a candidate compound. The dissolution of the complex by the candidate compound correlates with the ability of the compound to disrupt or inhibit the interaction between cryopyrin and the other peptide.

Another technique for drug screening provides high throughput screening for compounds having suitable binding affinity to cryopyrin peptides and is described in detail in WO 84/03564, incorporated herein by reference. Briefly, large numbers of different small peptide test compounds are synthesized on a solid substrate, such as plastic pins or some other surface. The peptide test compounds are then reacted with cryopyrin peptides and washed. Bound cryopyrin peptides are then detected by methods well known in the art.

Another technique uses cryopyrin antibodies, generated as discussed above. Such antibodies capable of specifically binding to cryopyrin peptides compete with a test compound for binding to cryopyrin. In this manner, the antibodies can be used to detect the presence of any peptide that shares one or more antigenic determinants of the cryopyrin peptide.

The present invention contemplates many other means of screening compounds. The examples provided above are presented merely to illustrate a range of techniques available. One of ordinary skill in the art will appreciate that many other screening methods can be used.

In particular, the present invention contemplates the use of cell lines transfected with cryopyrin and variants thereof for screening compounds for activity, and in particular to high throughput screening of compounds from combinatorial libraries (e.g., libraries containing greater than 10⁴ compounds). The cell lines of the present invention can be used in a variety of screening methods. In some embodiments, the cells can be used in second messenger assays that monitor signal transduction following activation of cell-surface receptors (e.g., activation of caspase-1). In other embodiments, the cells can be used in reporter gene assays that monitor cellular responses at the transcription/translation level. In still further embodiments, the cells can be used in cell proliferation assays to monitor the overall growth/no growth response of cells to external stimuli.

In second messenger assays, the host cells are preferably transfected as described above with vectors encoding cryopyrin or variants or mutants thereof. The host cells are then treated with a compound or plurality of compounds (e.g., from a combinatorial library) and assayed for the presence or absence of a response (e.g., activation of caspase-1). It is contemplated that at least some of the compounds in the combinatorial library can serve as agonists, antagonists, activators, or inhibitors of the protein or proteins encoded by the vectors. It is also contemplated that at least some of the compounds in the combinatorial library can serve as agonists, antagonists, activators, or inhibitors of protein acting upstream or downstream of the protein encoded by the vector in a signal transduction pathway.

In some embodiments, the second messenger assays measure fluorescent signals from reporter molecules that respond to intracellular changes (e.g., Ca²⁺ concentration, membrane potential, pH, IP₃, cAMP, arachidonic acid release) due to stimulation of membrane receptors and ion channels (e.g., ligand gated ion channels; see Denyer et al., Drug Discov. Today 3:323 [1998]; and Gonzales et al., Drug. Discov. Today 4:431-39 [1999]). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the compound. Responses of the host cells to treatment with the compounds can be detected by methods known in the art, including, but not limited to, fluorescence microscopy, confocal microscopy (e.g., FCS systems), flow cytometry, microfluidic devices, FLIPR systems (See, e.g., Schroeder and Neagle, J. Biomol. Screening 1:75 [1996]), and plate-reading systems. In some preferred embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The cells are also useful in reporter gene assays. Reporter gene assays involve the use of host cells transfected with vectors encoding a nucleic acid comprising transcriptional control elements of a target gene (i.e., a gene that controls the biological expression and function of a disease target) spliced to a coding sequence for a reporter gene. Therefore, activation of the target gene results in activation of the reporter gene product. In some embodiments, the reporter gene construct comprises the 5′ regulatory region (e.g., promoters and/or enhancers) of a protein whose expression is controlled by cryopyrin or cryopyrin signaling partners in association with a reporter gene. Examples of reporter genes finding use in the present invention include, but are not limited to, chloramphenicol transferase, alkaline phosphatase, firefly and bacterial luciferases, β-galactosidase, β-lactamase, and green fluorescent protein. The production of these proteins, with the exception of green fluorescent protein, is detected through the use of chemiluminescent, calorimetric, or bioluminescent products of specific substrates (e.g., X-gal and luciferin). Comparisons between compounds of known and unknown activities may be conducted as described above.

II. Modulation of Cryopyrin Signaling

In some embodiments, the present invention provides therapeutics useful in the treatment of inflammatory disease or otherwise modulating immune responses. In other embodiments, the present invention provides methods of enhancing immune responses to vaccines (e.g. via adjuvants). Such compounds find use in the modulation of cryopyrin signaling (e.g., compounds that increase or decrease cryopyrin signaling via activation of caspase-1). In some embodiments, cryopyrin modulators are cryopyrin nucleic acids or proteins. In other embodiments, cryopyrin modulators are cryopyrin activity or expression modulators (e.g., identified using the drug screens described above).

A. Antisense and RNAi

In some embodiments, the present invention targets the expression of cryopyrin modulating proteins (e.g., repressors). For example, in some embodiments, the present invention employs compositions comprising oligomeric antisense compounds, particularly oligonucleotides, for use in modulating the function of nucleic acid molecules encoding cryopyrin modulating proteins, ultimately modulating (e.g., increasing) the amount of cryopyrin protein expressed. This is accomplished by providing antisense compounds (e.g., antisense oligonucleotides, siRNA, etc.) that specifically hybridize with one or more nucleic acids encoding cryopyrin modulating proteins. The specific hybridization of an oligomeric compound with its target nucleic acid interferes with the normal function of the nucleic acid.

i. RNA Interference (RNAi)

In some embodiments, RNAi is utilized to inhibit cryopyrin modulating protein function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:4948; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Corners, synthesised using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 August 1; 31(15):4417-24, each of which is herein incorporated by reference in its entirety. In addition, software (e.g., the MWG online siMAX siRNA design tool) is commercially or publicly available for use in the selection of siRNAs.

ii. Antisense

In other embodiments, the present invention employs compositions comprising oligomeric antisense compounds, particularly oligonucleotides (e.g., those identified in the drug screening methods described below), for use in modulating the function of nucleic acid molecules encoding cryopyrin modulating proteins, ultimately modulating the amount of cryopyrin expressed. This is accomplished by providing antisense compounds that specifically hybridize with one or more nucleic acids encoding cryopyrin modulating proteins. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cryopyrin modulating proteins. In the context of the present invention, “modulation” means either an increase (stimulation) or a decrease (inhibition) in the expression of a gene. For example, expression may be inhibited to potentially prevent tumor metastasis.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a cryopyrin modulating protein. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]2, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. Antibodies

In other embodiments, the present invention provides antibodies that target cryopyrin modulating proteins or cryopyrin signal pathway components. In preferred embodiments, the antibodies used for therapy are humanized antibodies. Methods and compositions for generating antibodies are described below.

C. Small Molecule Drugs

In still further embodiments, the present invention provides drugs (e.g., small molecule drugs) that enhance cryopyrin activity (e.g., by inhibiting cryopyrin modulating proteins or enhancing cryopyrin expression or activity (e.g., activation of caspase-1). In some embodiments, small molecule drugs are identified using the drug screening methods described above.

D. Adjuvants

In still other embodiments, the present invention provides methods of enhancing the immune response to vaccines. In some embodiments, the present invention provides methods of enhancing adjuvant activity by increasing cryopyrin activity (e.g., using the cryopyrin modulators described herein). In some embodiments, the adjuvants include, but are not limited to Imiquimod and Resiquimod.

E. Genetic And Transplantation Therapies

In yet other embodiments, the present invention contemplates the use of any genetic manipulation for use in modulating the expression of cryopyrin or cryopyrin modulating proteins or signaling partners of cryopyrin. Examples of genetic manipulation include, but are not limited to, delivery of cryopyrin (e.g., to cells, tissues, or subjects). Delivery of nucleic acid construct to cells in vitro or in vivo may be conducted using any suitable method. A suitable method is one that introduces the nucleic acid construct into the cell such that the desired event occurs (e.g., expression of an antisense construct). For example, cells may be transfected ex vivo to increase cryopyrin expression and the transfected cells may be transplanted to the site of interest.

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Appl. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

III. Generation of Cyropyrin Antibodies

Antibodies can be generated to allow for the detection of cyropyrin protein (e.g., in drug screening or research embodiments of the present invention). The antibodies may be prepared using various immunogens. In one embodiment, the immunogen is a human cyropyrin peptide to generate antibodies that recognize human cryopyrin. Such antibodies include, but are not limited to polyclonal, monoclonal, chimeric, single chain, Fab fragments, and Fab expression libraries.

Various procedures known in the art may be used for the production of polyclonal antibodies directed against cryopyrin. For the production of antibody, various host animals can be immunized by injection with the peptide corresponding to the cryopyrin epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward cryopyrin, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.). These include but are not limited to the hybridoma technique originally developed by Kohler and Milstein (Kohler and Milstein, Nature 256:495-497 [1975]), as well as the trioma technique, the human B-cell hybridoma technique (See e.g., Kozbor et al., Immunol. Tod., 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In an additional embodiment of the invention, monoclonal antibodies are produced in germ-free animals utilizing technology such as that described in PCT/US90/02545). Furthermore, it is contemplated that human antibodies will be generated by human hybridomas (Cote et al., Proc. Natl. Acad. Sci. USA 80:2026-2030 [1983]) or by transforming human B cells with EBV virus in vitro (Cole et al., in Monoclonal Antibodies and Cancer Therapy, Alan R. Liss, pp. 77-96 [1985]).

In addition, it is contemplated that techniques described for the production of single chain antibodies (U.S. Pat. No. 4,946,778; herein incorporated by reference) will find use in producing cryopyrin specific single chain antibodies. An additional embodiment of the invention utilizes the techniques described for the construction of Fab expression libraries (Huse et al., Science 246:1275-1281 [1989]) to allow rapid and easy identification of monoclonal Fab fragments with the desired specificity for cryopyrin. In some embodiments, humanized antibodies are generated (See e.g., U.S. Pat. Nos. 6,180,370, 5,585,089, 6,054,297, and 5,565,332; each of which is herein incorporated by reference).

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g, gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. (As is well known in the art, the immunogenic peptide should be provided free of the carrier molecule used in any immunization protocol. For example, if the peptide was conjugated to KLH, it may be conjugated to BSA, or used directly, in a screening assay).

The foregoing antibodies can be used in methods known in the art relating to the localization and structure of cryopyrin (e.g., for Western blotting), measuring levels thereof in appropriate biological samples, etc. The antibodies can be used to detect cryopyrin in a biological sample from an individual. The biological sample can be a biological fluid, such as, but not limited to, blood, serum, plasma, interstitial fluid, urine, cerebrospinal fluid, and the like, containing cells.

The biological samples can then be tested directly for the presence of human cryopyrin using an appropriate strategy (e.g., ELISA or radioimmunoassay) and format (e.g., microwells, dipstick (e.g., as described in International Patent Publication WO 93/03367), etc. Alternatively, proteins in the sample can be size separated (e.g., by polyacrylamide gel electrophoresis (PAGE), in the presence or not of sodium dodecyl sulfate (SDS), and the presence of cryopyrin detected by immunoblotting (Western blotting). Immunoblotting techniques are generally more effective with antibodies generated against a peptide corresponding to an epitope of a protein, and hence, are particularly suited to the present invention.

Another method uses antibodies as agents to alter signal transduction. Specific antibodies that bind to the binding domains of cryopyrin or other proteins involved in intracellular signaling can be used to inhibit the interaction between the various proteins and their interaction with other ligands. Antibodies that bind to the complex can also be used therapeutically to inhibit interactions of the protein complex in the signal transduction pathways leading to the various physiological and cellular effects of NF-κB. Such antibodies can also be used diagnostically to measure abnormal expression of cryopyrin, or the aberrant formation of protein complexes, which may be indicative of a disease state.

IV. Transgenic Animals Expressing Exogenous Cryopyrin Genes and Homologs, Mutants, and Variants Thereof

The present invention contemplates the generation of transgenic animals comprising an exogenous cryopyrin gene or homologs, mutants, or variants thereof. In preferred embodiments, the transgenic animal displays an altered phenotype as compared to wild-type animals. In some embodiments, the altered phenotype is the overexpression of mRNA for a cryopyrin gene as compared to wild-type levels of cryopyrin expression. In other embodiments, the altered phenotype is the decreased expression of mRNA for an endogenous cryopyrin gene as compared to wild-type levels of endogenous cryopyrin expression. Methods for analyzing the presence or absence of such phenotypes include Northern blotting, mRNA protection assays, and RT-PCR. In other embodiments, the transgenic mice have a knock out mutation of the cryopyrin gene. In still further embodiments, the transgenic animal comprises a variant cryopyrin gene. In preferred embodiments, the transgenic animals display a disease phenotype (e.g., an inflammatory disease).

The transgenic animals of the present invention find use in dietary and drug screens. In some embodiments, the transgenic animals (e.g., animals lacking a functional cryopyrin gene) are fed test or control diets and the response of the animals to the diets is evaluated. In other embodiments, test compounds (e.g., a drug that is suspected of being useful to treat inflammatory disease or as an adjuvant) and control compounds (e.g., a placebo) are administered to the transgenic animals and the control animals and the effects evaluated. In other embodiments, cryopyrin (e.g., cryopyrin encoding nucleic acids or cryopyrin polypeptides) are administered to animals lacking a functional cryopyrin gene and the effect on immune response is assayed.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter, which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260-1264 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82:6927-693 [1985]). Transfection is easily and efficiently obtained by culturing the blastomeres on a monolayer of virus-producing cells (Van der Putten, supra; Stewart, et al, EMBO J., 6:383-388 [1987]). Alternatively, infection can be performed at a later stage. Virus or virus-producing cells can be injected into the blastocoele (Jahner et al., Nature 298:623-628 [1982]). Most of the founders will be mosaic for the transgene since incorporation occurs only in a subset of cells that form the transgenic animal. Further, the founder may contain various retroviral insertions of the transgene at different positions in the genome, which generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154-156 [1981]; Bradley et al., Nature 309:255-258 [1984]; Gossler et al., Proc. Acad. Sci. USA 83:9065-9069 [1986]; and Robertson et al., Nature 322:445-448 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468-1474 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants. Methods for homologous recombination are described in U.S. Pat. No. 5,614,396, incorporated herein by reference.

V. Pharmaceutical Compositions Containing cryopyrin Analogs and Modulators

The present invention further provides pharmaceutical compositions which may comprise all or portions of cryopyrin ligands, inhibitors, activators or antagonists of cryopyrin bioactivity, including antibodies, alone or in combination with at least one other agent, such as a stabilizing compound, and may be administered in any sterile, biocompatible pharmaceutical carrier, including, but not limited to, saline, buffered saline, dextrose, and water.

As is well known in the medical arts, dosages for any one patient depends upon many factors, including the patient's size, body surface area, age, the particular compound to be administered, sex, time and route of administration, general health, and interaction with other drugs being concurrently administered.

Accordingly, in some embodiments of the present invention, cryopyrin modulators or ligands can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of a compound of the present invention may be that amount that suppresses symptoms of an inflammatory disease. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations that can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition. For polynucleotide or amino acid sequences of cryopyrin, conditions indicated on the label may include treatment of conditions related to inflammation.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range that adjusts levels of the pharmaceutical of interest.

A therapeutically effective dose refers to that amount of a compound of the present invention that ameliorates symptoms of the disease state. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference). Those skilled in the art may employ different formulations for activators of cryopyrin than for the inhibitors of cryopyrin. Administration to the bone marrow may necessitate delivery in a manner different from intravenous injections.

### EXPERIMENTAL

The following examples are provided in order to demonstrate and further illustrate certain preferred embodiments and aspects of the present invention and are not to be construed as limiting the scope thereof.

### Example 1

This Example demonstrates that cryopyrin is necessary for modulating certain types of immune responses.

A. Methods

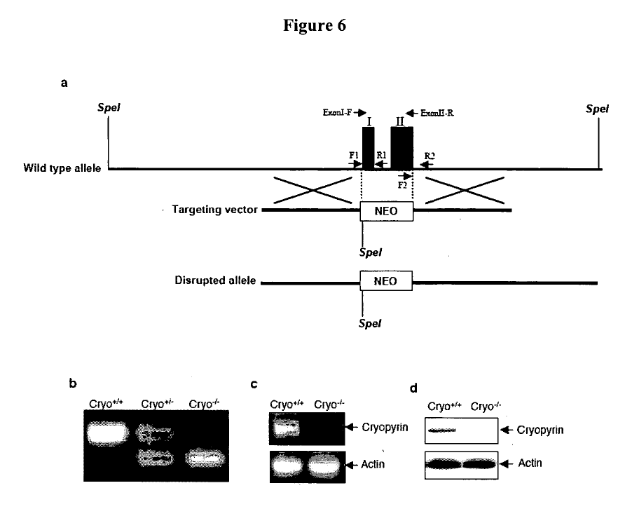

Mice. Cryopyrin knock-out (KO) mice were obtained from Millennium Pharmaceuticals and were generated by homologous recombination in ES cells by replacement of exon I and II of the cryopyrin gene encoding the N-terminal Pyrin domain with an IRES-β-gal-neomycin-resistance cassette via a targeting vector (FIG. 6). A positive ES clone was used to generate chimeric mice. 129/C57BL/6 chimeric mice were crossed with C57BL/6 females to generate heterozygous mice. Cryopyrin KO and WT mice were generated by crossing male and female heterozygous mice. TLR7, MyD88 and ASC KO mice have been described (Hemmi et al., Nat Immunol 3, 196-200 (2002)).

Microbial ligands and antibodies. Ultrapure LPS was from E. coli 0111:B4, Pam2CGDPKHPKSF (FSL-1), Pam3CSKKK4, lipid A, purified flagellin from S. typhimurium, R837, and CpG oligonucleotide (5′-TCCATGACGTTCCTGACGTT-3′ (SEQ ID NO:1) were purchased from Invivogen and muramyl dipeptide from Bachem. Purified total RNA from E. coli and mouse liver was purchased from Ambion. Total RNA from L. pneumophila and L. monocytogenes was prepared using a RiboPure-Bacteria kit from Ambion and RNAse was from Promega. Absorbance260/280 of RNAs was 1.7/2. Rabbit anti9 mouse caspase-1 and cryopyrin antibodies have been described (O'Connor et al., J Immunol 171, 6329-33 (2003); Lamkanfi et al., J Biol Chem 279, 24785-93 (2004)). The antibodies for mouse Iκ-Bα, phospho-Iκ-Bα, p38 and phospho-p38 were from Cell Signaling.

Western Blotting. Cell extracts were prepared. For analysis of caspase-1 activation, macrophages were cultured with ligands for 1-3 h and then with medium containing 5 mM ATP (Sigma) for 30 min. Extracts were prepared from cells and culture supernatants by adding lysis buffer containing 1% NP-40 supplemented with complete protease inhibitor cocktail (Roche, Mannheim, Germany) and 2 mM dithiotheitol, separated by SDS-PAGE and transferred to nitrocellulose membranes. Membranes were immunoblotted with primary antibodies and proteins detected with appropriate secondary anti-rabbit antibody conjugated to horseradish peroxidase followed by enhanced chemiluminescence.

Measurements of cytokines. BMDM and peritoneal macrophages were prepared as previously described. Cells were stimulated with various microbial and synthetic ligands for 24 h and the supernatants were analyzed for IL-1β, IL-18, TNFα and IL-6 secretion. The following ligand concentrations were used: FLS-1, Pam3CSKKK4, LPS, lipoteichoic acid, lipid A and MDP were used at 1 μg ml⁻¹. For analysis of cooperation of LPS with various ligands, 10 ng ml⁻¹ of LPS and 10 ng ml⁻¹ of various ligands were used. In some experiments, the macrophages were stimulated with ligands for 16 h and then with medium containing 2 mM ATP (Sigma) for 30 min, washed, and cultured for an additional 5 h. Mouse cytokines were measured in culture supernatants with enzyme-linked immunoabsorbent assay (ELISA) kits (R and D Systems, Minneapolis, Minn.).

Immunization of mice. Mice were immunized with R837 (100 μg) or CpG (100 μg) and human serum albumin (HSA) (50 μg) by intraperitoneal injection. Mice were boosted with R837 (20 μg) or CpG (20 μg) and HSA (10 μg) three weeks later. Serum sample were obtained two weeks after the first immunization and one week after the boost, and antigen specific serum immunoglobulin was assessed by ELISA using antibodies against mouse immunoglobulin and isotype controls (Southern Biotechnology).

B. Results

To define the role of cryopyrin in inflammatory responses, cryopyrin deficient mice were generated by homologous recombination using a targeting construct to replace the exons I and II encoding the Pyrin domain of cryopyrin that is essential for effector function of the protein (FIG. 6). Cryopyrin−/− mice were fertile and appeared healthy when housed under standard specific pathogen-free environment.

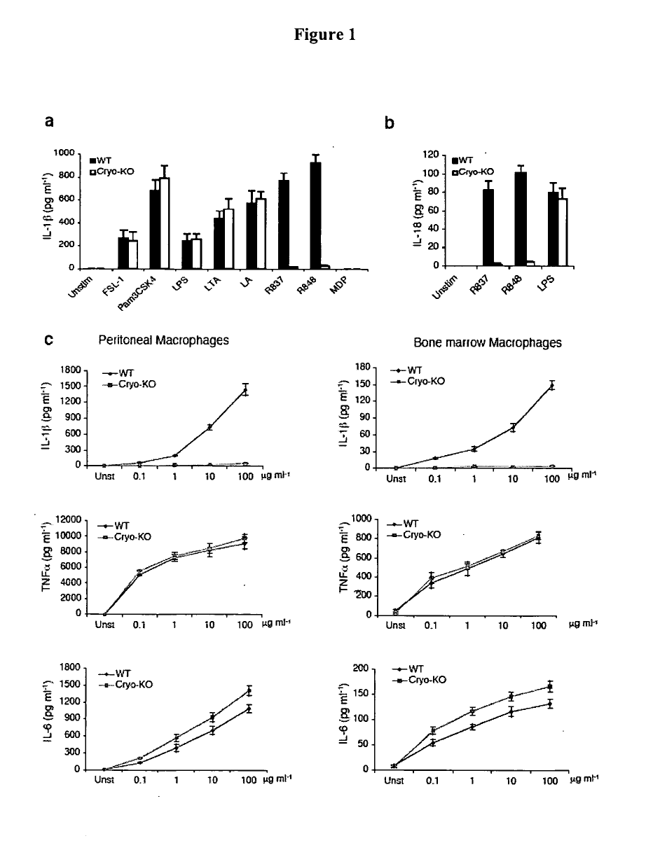

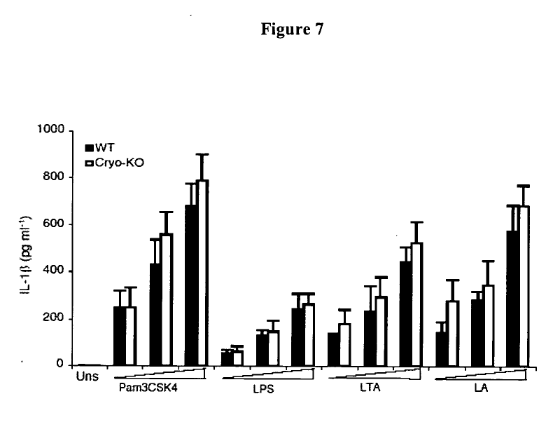

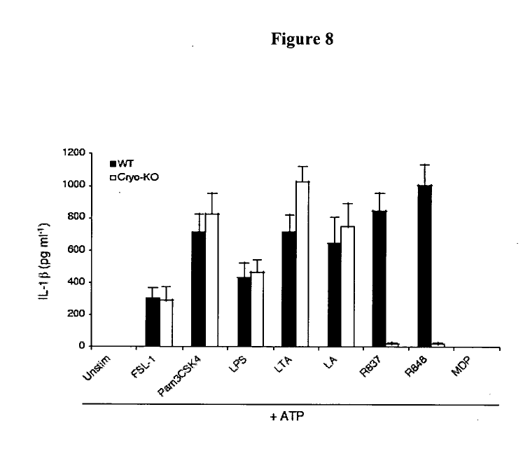

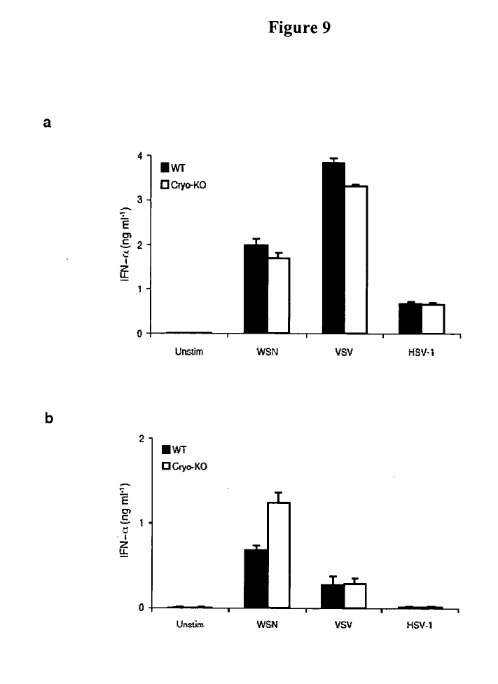

The role of cryopyrin in caspase-1-dependent IL-1β secretion was investigated using thioglycollate-elicited peritoneal macrophages and bone marrow-derived macrophages (BMDM) and multiple bacterial and synthetic ligands. Stimulation of peritoneal macrophages or BMDM with several TLR2 and TLR4 agonists including diacylated (Pam2CGDPKHPHSF) and triacylated (Pam3CSK4) synthetic lipopeptides, lipoteichoic acid, lipopolysaccharide (LPS) and lipid A induced comparable levels of IL-1β in wild-type (WT) and cryopyrin−/− macrophages (FIG. 1a and FIG. 7). Similar results were obtained when macrophages were stimulated with bacterial ligands and treated briefly with ATP (FIG. 8), a signal that enhances the secretion of IL-1β in pre-stimulated macrophages (Perregaux et al., J Immunol 165, 4615-23 (2000)). Incubation of macrophages with muramyl dipeptide (MDP) did not induce secretion of IL-1β over background levels in WT and cryopyrin−/− macrophages even after addition of ATP (FIG. 1a; FIG. 8). Furthermore, production of interferon-α induced by several viruses was unimpaired in macrophages and dendritic cells from cryopyrin−/− mice (FIG. 9). Secretion of IL-1β and IL-18 induced by the low molecular weight imidazoquinoline compounds Imiquimod (R837) and Resiquimod (R848), that are known to activate pro-inflammatory responses in the mouse through TLR7 (Hemmi et al., Nat Immunol 3, 196-200 (2002); Jurk et al., Nat Immunol 3, 499 (2002)) was abrogated in both peritoneal macrophages and BMDM from cryopyrin−/− mice (FIG. 1a, b, c). In contrast, cryopyrin was dispensable for the production of the pro4 inflammatory cytokines TNFα and IL-6 induced by R837 stimulation (FIG. 1c). These results indicate that cryopyrin is specifically required for the secretion of IL-1β and IL-18 induced by the synthetic molecules R837 and R848.

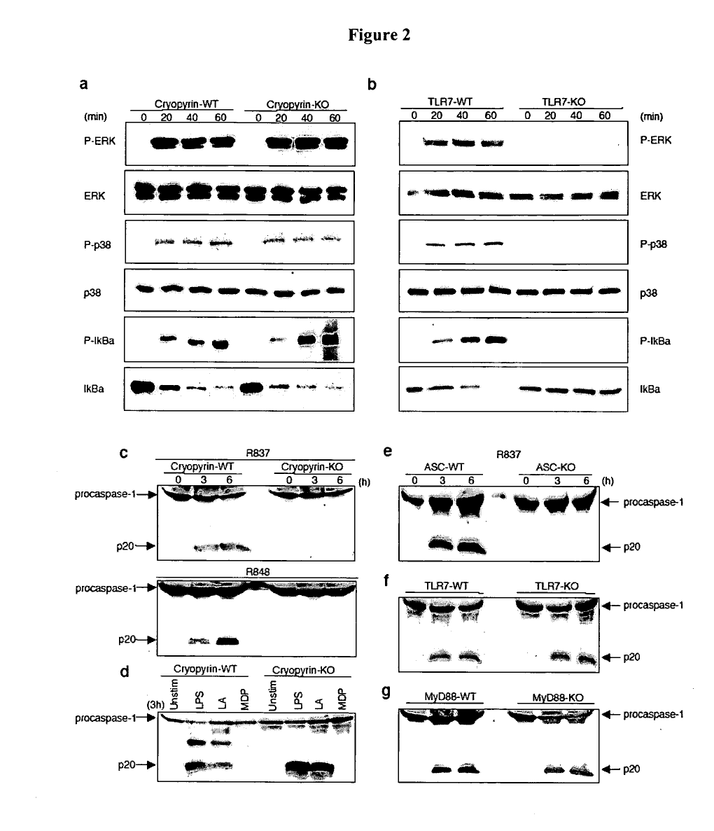

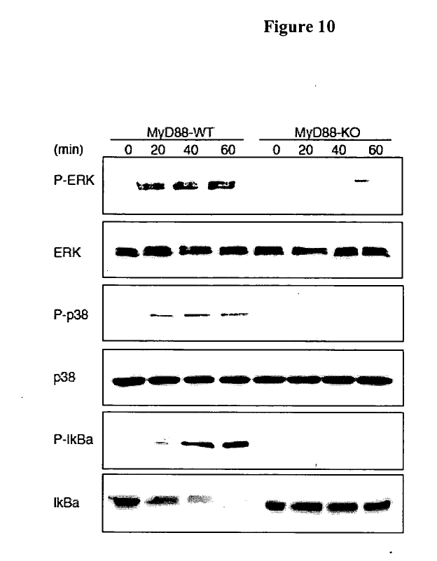

The induction of IL-1β secretion is thought to involve the upregulation of pro-IL-1β through transcriptional mechanisms via NF-κB and then a second stimulus that leads to the activation of caspase-1, processing of pro-IL-1β, and release of mature IL-1β (Dinarello et al., Ann N Y Acad Sci 856, 1-11 (1998); Perregaux et al., J Immunol 165, 4615-23 (2000)). It was found that stimulation with R837 induced comparable levels of NF-κB, ERK and p38 activation in WT and cryopyrin−/− macrophages (FIG. 2a). By contrast, the activation of NF-κB and MAPKs was abolished in TLR7- and MyD88-deficient macrophages (FIG. 2b; FIG. 10). Proteolytic processing of pro-caspase-1 was induced in WT macrophages by both R837 and R848, as determined by the detection of the mature 20 kDa subunit of caspase-1 (FIG. 2c). Such activation of caspase-1 was abrogated in macrophages lacking cryopyrin (FIG. 2c) or ASC (FIG. 2e), an adaptor that links cryopyrin to caspase-1 (Agostini et al., Immunity 20, 319-25 (2004); Dowds et al., J Biol Chem 279, 21924-8 (2004)). In contrast, activation of caspase-1 was unimpaired in cryopyrin−/− macrophages in response to LPS and lipid A (FIG. 2d), further indicating that the ligand-recognition function of cryopyrin is highly specific. MDP did not induce proteolytic processing of pro-caspase-1 in mouse macrophages (FIG. 2d), consistent with its inability to induce IL-1β secretion (FIG. 1a). Activation of caspase-1 induced by R837 proceeded normally in TLR7- or MyD88-deficient macrophages (FIG. 2f, g). These results demonstrate that cryopyrin is essential for caspase-1 processing independent of NF-κB and MAPK activation in response to R837 and R848. Furthermore, TLR7 and MyD88 are required for NF-κB and MAPK activation but dispensable for caspase-1 activation.

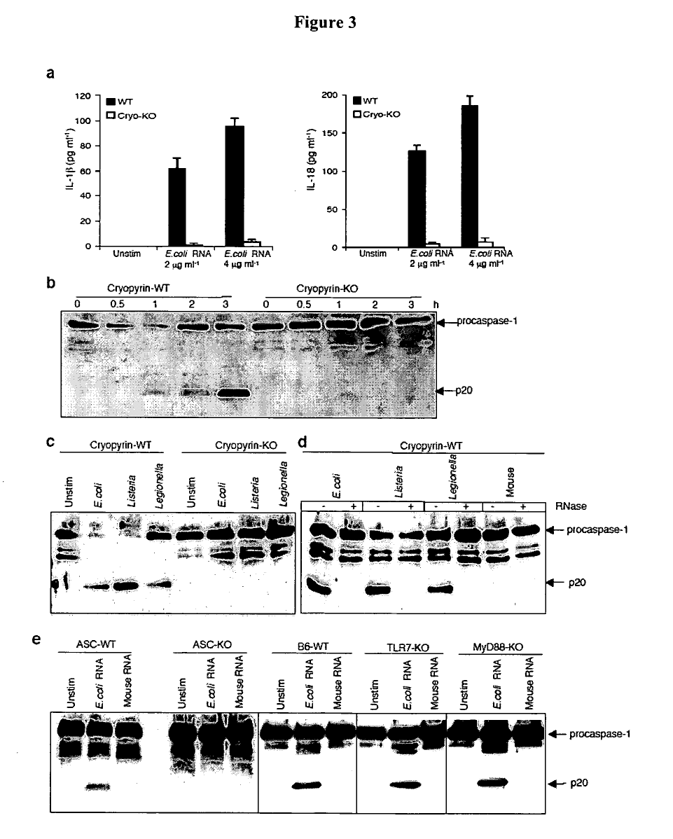

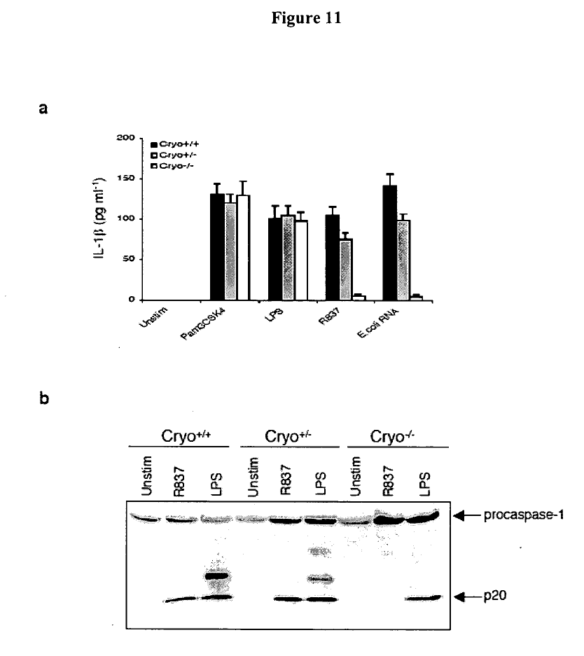

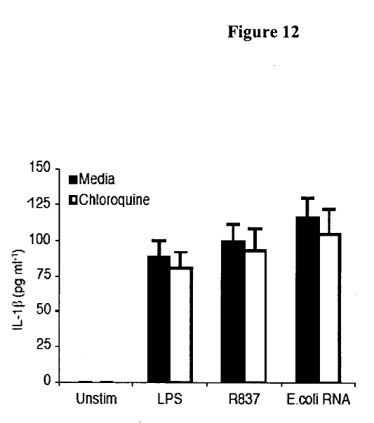

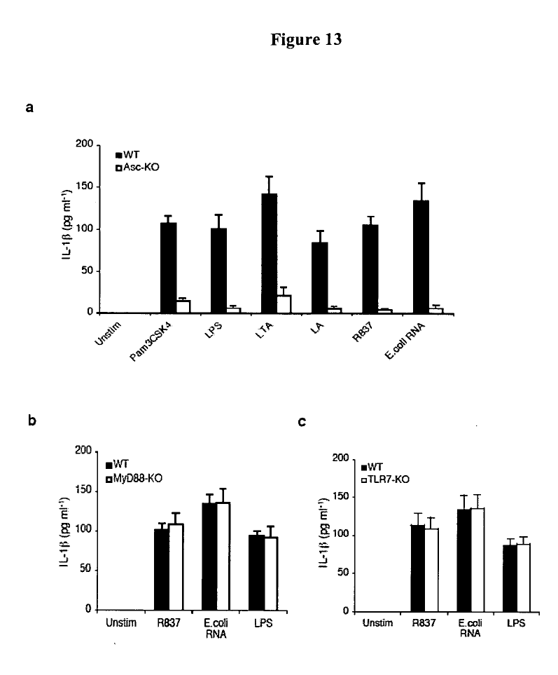

R837 and R848 structurally resemble purine bases (Dockrell et al., J Antimicrob Chemother 48, 751-5 (2001)). Secretion of IL-1 and IL-18 was induced by Escherichia coli RNA in WT and cryopyrin+/− macrophages, but this was abolished in cryopyrin−/− macrophages (FIG. 3a and FIG. 11). Treatment with chloroquine did not affect the production of IL-1β induced by bacterial RNA (FIG. 12). E. coli RNA induced rapid activation of caspase-1 in WT and cryopyrin+/− macrophages, but not in cryopyrin−/− macrophages (FIG. 3b). As was found with E. coli RNA, stimulation with total RNA from two additional bacteria Listeria monocytogenes, and Legionella pneumophila, but not total RNA from mouse liver, induced activation of caspase-1 in WT, but not in cryopyrin−/− macrophages (FIG. 3c, d). Treatment of the RNA preparations with RNase abolished their ability to induce activation of caspase-1 (FIG. 3d), indicating that RNA, but not a contaminant, triggers caspase-1 activation. Furthermore, secretion of IL-1β and processing of pro-caspase-1 induced by bacterial RNA was abrogated in macrophages lacking ASC, but this was unimpaired in macrophages deficient in TLR7- or MyD88-deficient macrophages (FIG. 3e and FIG. 13). These results demonstrate that bacterial RNA activates caspase-1 and IL-1β secretion and these events are mediated through cryopyrin and ASC independent of TLR7 and MyD88.

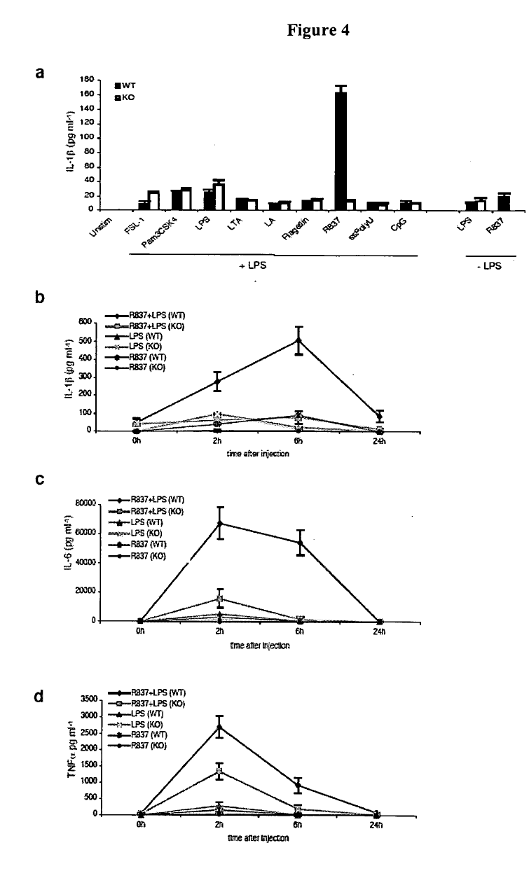

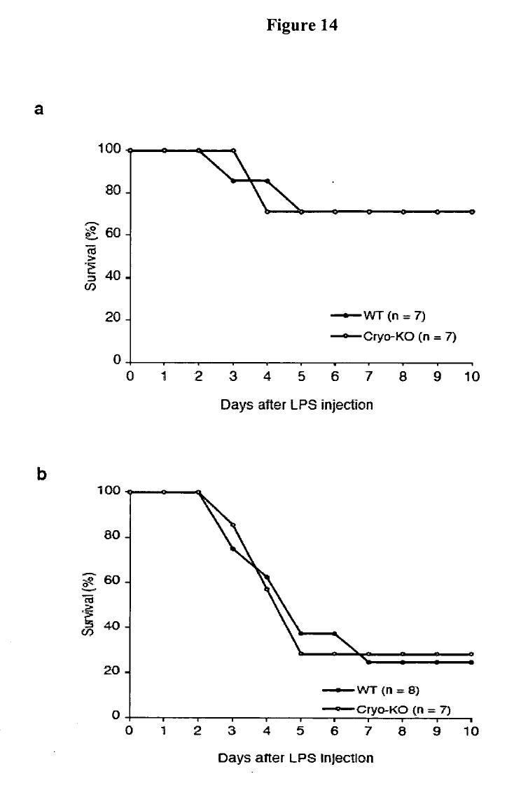

Mononuclear cells from patients with autoinflammatory syndromes spontaneously secrete IL-1β and IL-18 and exhibit enhanced production of IL-1β and IL-18 in response to low amounts of LPS (Agostini et al., supra; Janssen et al., Arthritis Rheum 50, 3329-33 (2004)). This is consistent with the observation that disease-associated cryopyrin mutations exhibit constitutive activity (Dowds et al., J Biol Chem 279, 21924-8 (2004)). Therefore, the ability of low doses of LPS to cooperate with low amounts of various microbial ligands in the production of IL-1β was investigated. LPS synergized with R837, but not with lipopeptides, lipoteichoic acid, lipid A, flagellin, or CpG oligodeoxynucleotide, for the secretion of IL-1β (FIG. 4a). Such enhancement of LPS-mediated IL-1β production by R837 was abrogated in cryopyrin−/− macrophages (FIG. 4a). The susceptibility to high doses of LPS induced comparable lethality in WT and cryopyrin−/− mice (FIG. 14). The present invention is not limited to a particular mechanism. Indeed, an understanding of the mechanism is not necessary to practice the present invention. Nonetheless, it is contemplated that the synergism between LPS and R837 may be explained, at least in part, by the ability of LPS to induce cryopyrin expression (O'Connor et al., J Immunol 171, 6329-33 (2003)). Consistent with the in vitro results, co-administration of R837 and LPS into mice induced higher levels of IL-1β in serum than injection of R837 or LPS alone (FIG. 4b). Furthermore, the enhancement of the LPS response by R837 was abolished in cryopyrin−/− mice (FIG. 4b). The serum levels of TNFα and IL-6 were also enhanced by co-injection of LPS and R837 when compared to those observed after administration of each molecule alone (FIG. 4c, d). Reduced levels of TNFα and IL-6 were detected in the serum of cryopyrin−/− mice after stimulation with LPS plus R837 (FIG. 4c, d), due to the induction of TNFα and IL-6 by IL-1β and/or IL-18 in vivo (Netea et al., Eur J Immunol 30, 3057-60 (2000)).

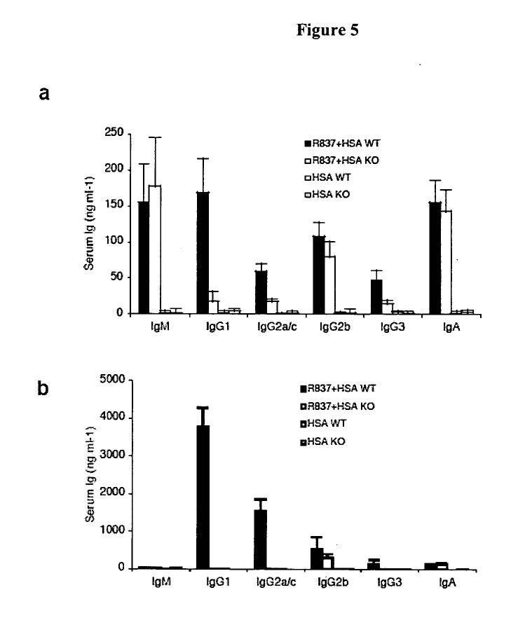

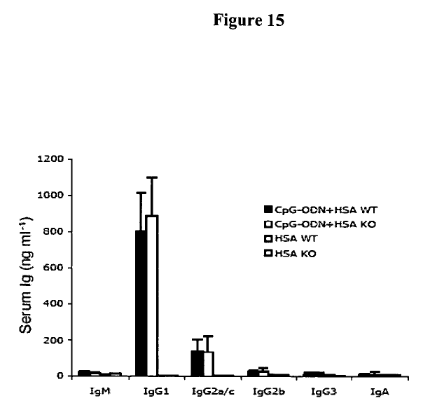

R837 and R848 are immune response modifiers with anti-viral and anti-tumor activity that are used clinically for the treatment of infectious and neoplastic diseases (Craft et al., J Immunol 175, 1983-90 (2005); Harrison et al., Antiviral Res 10, 209-23 (1988); Tomai et al., Antiviral Res 28, 253-64 (1995); Marks et al., J Am Acad Dermatol 44, 807-13 (2001)). To determine the role of cryopyrin in adaptive immune responses, the ability of R837 to serve as an adjuvant for the production of IgG against HSA, a T-cell dependent antigen was tested. Cryopyrin−/− animals exhibited a defect in the production of antigen specific IgG1, IgG2a/c, IgG3 after immunization with HSA and R837 (FIG. 5a). Levels of HSA-specific IgM, IgG2b and IgA were comparable in WT and cryopyrin−/− mice (FIG. 5a). After boosting with R837 and HSA three weeks later, there was a large increase in the levels of HSA-specific IgG1 and IgG2a/c in WT mice (FIG. 5b). Cryopyrin−/− mice exhibited a severe deficiency in the production of HSA-specific IgG1 and IgG2a/c after boosting (FIG. 5b). Enhancement of HSA-specific IgG responses by CpG was unaffected by cryopyrin deficiency (FIG. 15). These results indicate that cryopyrin is essential for R837-mediated adjuvant activity and is able to activate adaptive immunity during the production of antibody to T-cell dependent antigens.

These studies demonstrate that cryopyrin plays a role in the secretion of IL-1β and IL-18 by controlling the activation of pro-caspase-1 in response to bacterial RNA and the synthetic compounds R837 and R848. Secretion of IL-1β and IL-18 was shown to be regulated by separate signaling pathways that are independently controlled by TLR and NOD-LRR proteins. The identification of cryopyrin as a sensor for bacterial RNA and the nucleoside-based analogues R837 and R848 indicate that TLR and NOD-LRR proteins can be activated by the same or similar microbial structures (Hemmi et al., supra; Heil et al., Science 303, 1526-9 (2004); Lund et al., Proc Natl Acad Sci USA 101, 5598-603 (2004); Diebold et al., Science 303, 1529-31 (2004)). Bacterial RNA may be derived from phagocytosed bacteria or lysis of bacteria in the extracellular space and transported into the host cytosol, leading to cryopyrin recognition and activation.

The present invention is not limited to a particular mechanism. Indeed, an understanding of the mechanism is not necessary to practice the present invention. Nonetheless, the mechanism by which cryopyrin and TLR7/TLR8 discriminates between microbial and endogenous RNA remains poorly understood, although differences in nucleoside modification and polyA-tail between bacterial and mammalian RNA may be involved (Kariko et al., Immunity 23, 165-75 (2005); Koski et al., J Immunol 172, 3989-93 (2004)). The present invention is not limited to a particular mechanism. Indeed, an understanding of the mechanism is not necessary to practice the present invention. Nonetheless, it is contemplated that the potent adjuvant, antiviral and antitumor activity exerted by the imidazoquinoline compounds is explained by their ability to activate both TLR and cryopyrin signaling. The identification of cryopyrin as a sensor of bacterial RNA for caspase-1 activation has implications for host defense and RNA-based vaccines as well as for the understanding of inflammatory diseases and in particular autoinflammatory syndromes.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A method of screening compounds, comprising
a) contacting a cell expressing cryopyrin with a test compound; and
b) comparing the level of caspase-1 activation in the presence of said test compound to the level in the absence of said test compound.

2. The method of claim 1, wherein said level of caspase-1 activation is higher in the presence of said test compound relative to the level in the absence of said test compound.

3. The method of claim 1, wherein said test compound is an adjuvant.

4. The method of claim 1, wherein said test compound is a drug.

5. The method of claim 1, wherein said test compound is selected from the group consisting of a nucleotide, a nucleoside, and a purine.

6. The method of claim 1, wherein said test compound is a mimetic of a molecule selected from the group consisting of a nucleic acid, a nucleotide, a nucleoside, and a purine.

7. The method of claim 1, wherein said measuring the level of caspase-1 activation comprising measuring the level of a cytokine selected from the group consisting of IL-1β and IL-18.

8. The method of claim 1, wherein said cell is in a non-human mammal.

9. A method of modulating an immune response, comprising contacting a cell with an immune modulator and a cryopyrin modulator.

10. The method of claim 9, wherein said cryopyrin modulator stimulates cryopyrin expression or activity.

11. The method of claim 9, wherein said cell is cryopyrin deficient.

12. The method of claim 11, wherein said cryopyrin modulator is selected from the group consisting of a cryopyrin nucleic acid and a cryopyrin polypeptide.

13. The method of claim 9, wherein said immune modulator is an adjuvant.

14. The method of claim 13, further comprising administering a vaccine to said cell.

15. The method of claim 14, wherein production of vaccine specific IgG is stimulated in said cell.

16. The method of claim 1, wherein said immune modulator is a pathogen.

17. The method of claim 16, wherein said immune modulator is bacterial RNA.

18. The method of claim 9, wherein caspase-1 is activated in said cell.

19. The method of claim 1, wherein said cell is in an organism.

20. The method of claim 19, wherein said organism lacks a functional cyropyrin gene.
